(19) United States
(12) Patent Application Publication
(10) Pub. No.: US 2014/0171365 A1
(43) Pub. Date: Jun. 19, 2014

(54) PEGYLATED HUMAN HDL PARTICLE AND PROCESS FOR PRODUCTION THEREOF

(71) Applicants: Alan R. Tall, Cresskill, NJ (US); Nan Wang, Flushing, NY (US)

(72) Inventors: Alan R. Tall, Cresskill, NJ (US); Nan Wang, Flushing, NY (US)

(73) Assignee: THE TRUSTEES OF COLUMBIA UNIVERSITY IN THE CITY OF NEW YORK, New York, NY (US)

(21) Appl. No.: 14/007,289

(22) PCT Filed: Mar. 23, 2012
(86) PCT No.: PCT/US2012/030401

§ 371 Date: Mar. 7, 2014

Related U.S. Application Data
(60) Provisional application No. 61/467,723, filed on Mar. 25, 2011.

(51) Int. Cl. C07K 14/775

(52) U.S. Cl. CPC C07K 14/775
USPC 514/7.4; 530/359; 514/1.1

(57) ABSTRACT

Compositions comprising pegylated high density lipoprotein (HDL) particles, methods for producing compositions comprising pegylated HDL particles, and methods of treating various diseases and conditions using pegylated HDL particles are provided.

This application claims priority of U.S. Provisional Application No. 61/467,723, filed Mar. 25, 2011, the entire content of which is hereby incorporated by reference herein.

Throughout this application various publications and published patents are referenced. Full citations for these publications are presented in a References section immediately before the claims. The disclosures of these publications in their entireties are hereby incorporated by reference into this application in order to more fully describe the state of the art to which this invention pertains.

The work disclosed herein was made with government support under grant No. Hl 54591 from the National Institutes of Health. Accordingly, the U.S. Government has certain rights in this invention.

### BACKGROUND OF THE INVENTION

Plasma HDL cholesterol levels are inversely correlated to the risk of cardiovascular disease, suggesting a protective role. In humans, reconstituted HDL particles have been tested in clinical trials for their potential anti-atherosclerotic properties and these studies have indeed shown to cause regression of coronary atherosclerosis.

Currently, purified human apolipoprotein A-I (apoA-I), either in the recombinant or native form, is used in combination with phospholipids and bile salts in order to generate recombinant human HDL particles. HDL particles are made up of proteins and fats which carry cholesterol to the liver, where cholesterol is removed from the body. While the amphipathic nature of bile salt facilitates recombinant HDL particle formation, the residual bile salt in the recombinant HDL preparation can cause adverse effects in humans and therefore limit the quantity of usable recombinant HDL particles. The initial levels of plasma HDL particles and maintenance of its sufficient therapy concentration over appropriate period of time could determine the efficacy of the HDL particle therapy.

The mechanism responsible for turnover of plasma HDL particles is still poorly defined. Liver and kidney are the major organs involved in regulation of HDL particle catabolism. If the clearance of plasma HDL particle or its apolipoproteins can be reduced, it is likely that sufficient therapeutic concentration could be achieved with a reduced dose of HDL particles.

### SUMMARY OF THE INVENTION

The subject invention provides a composition comprising pegylated high-density lipoprotein (HDL) particles which comprise apolipoprotein A-I (ApoA-I), wherein at least 50% of the ApoA-I in the composition are monopegylated ApoA-I.

The subject invention also provides a composition comprising pegylated high-density lipoprotein (HDL) particles which comprises pegylated proteins other than pegylated ApoA-1.

The subject invention also provides a process for preparing a composition comprising pegylated HDL particles, the process comprising: (a) admixing HDL particles with a source of polyethylene glycol to form a mixture; (b) incubating the mixture from step (a) under conditions allowing for formation of a covalent bond between a protein of the HDL particles and a molecule of polyethylene glycol; and (c) separating pegylated HDL particles from the product of step (b), so as to thereby prepare the composition comprising pegylated HDL particles.

The subject invention also provides a composition prepared by the processes as described herein.

The subject invention also provides a method of treating a subject afflicted with an inflammatory vascular disease comprising administering to the subject an amount of the composition described herein, effective to treat the subject.

The subject invention also provides a method of treating a subject afflicted with dyslipidemia comprising administering to the subject an amount of the composition described herein, effective to treat the subject.

The subject invention also provides a method of increasing plasma HDL levels in a subject comprising administering to the subject an amount of the composition described herein, effective to increase plasma HDL levels in the subject.

The subject invention also provides a method of promoting cholesterol efflux from macrophage foam cells in a subject comprising administering to the subject an amount of the composition described herein, effective to promote cholesterol efflux from macrophage foam cells in the subject.

The subject invention also provides a method of decreasing the amount of white blood cells in a subject comprising administering to the subject an amount of the composition described herein, effective to decrease the amount of white blood cells in the subject.

The subject invention also provides the compositions described herein for use in treating a subject afflicted with an inflammatory vascular disease. The subject invention also provides the compositions described herein for use in treating a subject afflicted with dyslipidemia. The subject invention also provides the compositions described herein for use in increasing plasma high-density lipoprotein levels in a subject. The subject invention also provides the compositions described herein for use in promoting cholesterol efflux from macrophage foam cells in a subject. The subject invention also provides the compositions described herein for use in decreasing the amount of white blood cells in a subject.

The subject invention also provides a method of administering a compound to a subject, the method comprising administering to the subject an amount of a composition comprising the compound bound to pegylated HDL particles, so as to thereby administer the compound to the subject.

The subject invention also provides a composition comprising a compound bound to pegylated HDL particles and a method of increasing the amount LCAT in a subject comprising administering to the subject an amount of a composition comprising LCAT bound to pegylated HDL particles, effective to increase the amount of LCAT in a subject.

The subject invention also provides a method of treating a subject afflicted with atherosclerosis comprising administering to the subject an amount of a composition comprising LCAT bound to pegylated HDL particles, effective to treat the subject.

The subject invention also provides a method of treating a subject afflicted with a type of a kidney disease or at risk for a type of kidney disease comprising administering to the subject an amount of a composition comprising LCAT or ApoL-1 WT bound to pegylated HDL particles, effective to treat the subject or reduce or eliminate the risk for the kidney disease in the subject.

The subject invention also provides a composition comprising a compound bound to pegylated HDL particles for use as a delivery vehicle administering the compound to a subject.

The subject invention also provides a composition comprising LCAT bound to pegylated HDL particles for use increasing the amount of LCAT in a subject.

The subject invention also provides a composition comprising LCAT bound to pegylated HDL particles for use treating a subject afflicted with atherosclerosis.

The subject invention also provides a composition comprising LCAT or ApoL-1 WT bound to pegylated HDL particles for use in treating a subject afflicted with a type of a kidney disease or for reducing or eliminating the risk for the kidney disease in the subject.

### BRIEF DESCRIPTION OF THE FIGURES

FIG. 1A and FIG. 1B: Pegylation of human apoA-I. Purified human apoA-I was pegylated without (A1, B1 or B2) or with (A2, B3, B4) activated PEG for ˜16 hours at 4° C. (FIG. 1A) or room temperature (FIG. 1B) and analyzed by SDS-PAGE and Coomassie Blue Staining. Lower temperature results in partial pegylation and higher temperature leads to multipegylation of apoA-I. Longer incubation at 4° C. also results in multipegylation (not shown).

FIG. 2A and FIG. 2B: Cholesterol efflux from macrophage cells. Mouse peritoneal macrophages were labeled with 50 μg/ml acetyl-LDL and 1 μCi/ml [³H]cholesterol incorporated into the acetyl-LDL preparation overnight. Cholesterol efflux was initiated by adding apoA-I or PEG-apoA-I at the indicated concentration after washing the cells. Percentage efflux was determined as media count/(media+cellular count)×100. The PEG-apoA-I used in FIG. 2A was prepared as in FIG. 1A (partial pegylation) and the PEG-apoA-I prepared as in FIG. 1B (multipegylation) was used for efflux shown in FIG. 2B.

FIG. 3: Pegylation of human HDL. Human HDL was incubated with or without activated polyethylene glycol. The unmodified native HDL or pegylated HDL was subjected to SDS-PAGE analysis. Shown is the Coomassie Brilliant Blue staining of the gel.

FIG. 4: Native and pegylated human HDL promote cholesterol efflux from macrophages. Murine macrophage-like RAW cells were labeled with [³H]cholesterol reconstituted in cell culture media containing 10% fetal bovine serum. After washing, cholesterol efflux was initiated with addition of indicated amount of HDL. The percentage efflux of cholesterol was determined as described.

FIG. 5: Unpegylated HDL turnover in vivo. 0.8 mg HDL protein was injected into mice via tail vein. An aliquot of blood was taken from the mice at indicated time point following injection. 0.1 μl of plasma from each sample was subjected to Western analysis and the results are shown.

FIG. 6: Pegylated HDL turnover in vivo. 1 mg PEG-HDL protein preparation was injected into mice via tail vein. An aliquot of blood was taken from the mice at indicated time point following injection. 0.1 μl of plasma from each sample was subjected to Western analysis and the results are shown.

FIG. 7: PEG-rHDL is more effective than rHDL at lowering total white blood cells (Wbcs) in WTD fed Apoe^{-/-} mice. (Example 5) Apoe^{-/-} mice fed a WTD for 9 weeks prior to receiving an infusion of saline, rHDL (40 mg/kg) or PEG-rHDL (40 mg/kg). 2 weeks later the mice received a second infusion (same conditions) and 6 days later total white blood cells were measured. *p<0.0 vs. saline.

FIG. 8: Monocytes and neutrophils were assessed via flow cytometry and converted to cells/μl using counts from the complete blood cell analyses. (Example 5) Data presented as mean±SEM. *P<0.05 vs. saline.

FIG. 9: Aortic arches were dissected, cleaned and stained with Oil Red O for lesion quantification. (Example 5) Data presented as percentage mean lesion area±SEM. *P<0.005 vs. saline.

FIG. 10: Examination of the ability of human PEG-HDL and Native HDL to affect Cholesterol efflux from macrophage foam cells at 5 hours and 24 hours (Example 6).

### DETAILED DESCRIPTION OF THE INVENTION

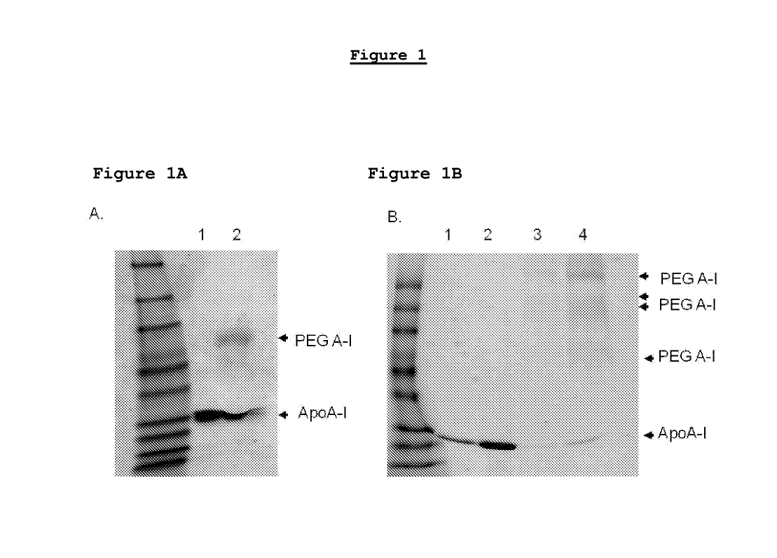

Applicants have previously developed a method to pegylate purified human apoA-I with activated methoxypoly(ethylene glycol) (mPEG) to increase the plasma half life of human apoA-I, which pegylated form of human apoA-I still retained its key biological activity, i.e., promotion of cholesterol efflux from macrophage foam cells (PCT International Application Publication No. WO 2010/141097). Although monopegylated species was observed using methods disclosed in WO 2010/141097, the reaction was incomplete as there was still significant native apoA-I in the preparation (FIG. 1A). When the reaction time or quantity of activated PEG were increased in order to provide more complete pegylation, a mixture of multipegylated species was obtained or underpegylation was observed (page 30, lines 14-20 of WO 2010/141097 and FIG. 1B). As a result, the method of WO 2010/141097 did not produce compositions containing more than 40%-50% monopegylated apoA-I.

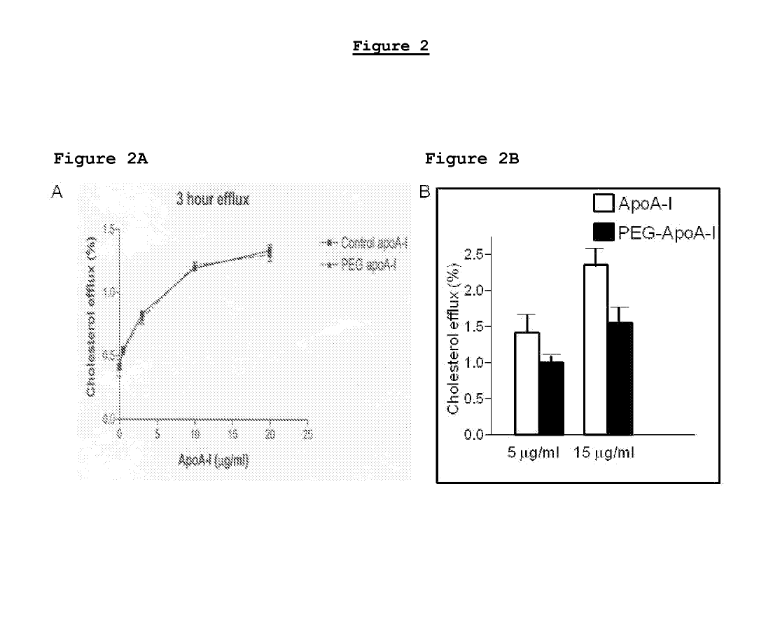

Since the monopegylated species is preferable over the native and the multipegylated apoA-I, which was demonstrated to have loss of function as shown by reduced capacity to promote cholesterol efflux from macrophage foam cells (page 33, lines 6-8 of WO 2010/141097 and FIG. 1B FIG. 2B), an improved pegylation process which can produce a higher concentration of monopegylated-apoA-I species is required.

As described herein, a novel method using HDL particles rather than its delipidated apolipoprotein as the substrate for protein pegylation has a number of advantages over the method according to WO 2010/141097.

Specifically, the pegylation method provided herein can produce HDL particle compositions containing more than 50% monopegylated apoA-I while being essentially free of the less desirable multipegylated species. Use of native HDL (apoA-I) as substrate for pegylation also increases the specificity and efficiency of targeted pegylation, since the essential amino acid residues of apoA-I required for lipidation to form HDL and function of HDL would likely be protected from pegylation by lipid molecules in HDL particles. Other advantages include improved consistency, cost savings (less material needed), absence of bile salts, and ease of separation (gradient centrifugation can be used). In comparison, the method disclosed in WO 2010/141097 had scale-up problems in part due to the difficulty of separation.

The subject invention provides a composition comprising pegylated high-density lipoprotein (HDL) particles which comprise apolipoprotein A-I (ApoA-I), wherein at least 50% of the ApoA-I in the composition are monopegylated ApoA-I. In one embodiment, at least 70% or at least 80% of the ApoA-I in the composition are monopegylated ApoA-I. In another embodiment, at least 90% of the ApoA-I in the composition are monopegylated ApoA-I. In yet another embodiment, at least 98% or at least 99% of the ApoA-I in the composition are monopegylated ApoA-I.

In one embodiment, the composition is essentially free of multipegylated ApoA-I. In another embodiment, the composition is essentially free of bile salts.

In one embodiment, at least 50% of the total monopegylated ApoA-I in the composition are N-terminally pegylated. In another embodiment, at least 60% of the total monopegylated ApoA-I in the composition are N-terminally pegylated. In another embodiment, at least 70% of the total monopegylated ApoA-I in the composition are N-terminally pegylated. In another embodiment, at least 80% of the total monopegylated ApoA-I in the composition are N-terminally pegylated. In another embodiment, about 80% of the total monopegylated ApoA-I in the composition are N-terminally pegylated.

In one embodiment, the monopegylated ApoA-I comprise a polyethylene glycol covalently bonded to the ApoA-I, which polyethylene glycol has a molecular weight of between 10,000 and 30,000 Daltons. In another embodiment, the polyethylene glycol has a molecular weight of between 15,000 and 25,000 Daltons. In another embodiment, the polyethylene glycol has a molecular weight of between 19,000 and 21,000 Daltons. In another embodiment, the polyethylene glycol has a molecular weight of about 20,000 Daltons.

In one embodiment, the pegylated HDL particle is pegylated recombinant HDL particle or pegylated mutant HDL particle. In another embodiment, the pegylated HDL particle is pegylated reconstituted HDL particle. In another embodiment, the pegylated HDL particle is pegylated native HDL particle. In yet another embodiment, the pegylated HDL particle is pegylated human HDL particle.

The subject invention also provides a composition comprising pegylated high-density lipoprotein (HDL) particles which comprises pegylated proteins other than pegylated ApoA-1. In one embodiment, the composition comprises pegylated ApoA-1 and a different pegylated protein. A In another embodiment, the composition comprises pegylated proteins, wherein the pegylated proteins include at least one of apolipoprotein A-II (apo A-II), apolipoprotein A-IV (ApoA-IV), apolipoprotein A-V (Apo V), apolipoprotein C-I (Apo C-I), apolipoprotein C-II (Apo C-II), apolipoprotein C-III (Apo C-III), apolipoprotein D (Apo D), apolipoprotein E (Apo E), apolipoprotein F (Apo F), apolipoprotein M (Apo M), lecithin cholesterol acyltransferase (LCAT), cholesteryl ester transfer protein (CETP), phospholipid transfer protein (PLTP), paraoxonase, platelet-activating factor acylhydrolase (PAF-AH), clusterin (apo J), serum amyloid A (SAA) and tissue factor pathway inhibitor (TFPI).

In one embodiment, at least 50% of the proteins in the composition are monopegylated proteins. In another embodiment, at least 70% or at least 80% of the proteins in the composition are monopegylated proteins. In another embodiment, at least 90% of the proteins in the composition are monopegylated proteins. In yet another embodiment, at least 98% or 99% of the proteins in the composition are monopegylated proteins.

The subject invention also provides a process for preparing a composition comprising pegylated HDL particles, the process comprising: (a) admixing HDL particles with a source of polyethylene glycol to form a mixture; (b) incubating the mixture from step (a) under conditions allowing for formation of a covalent bond between a protein of the HDL particles and a molecule of polyethylene glycol; and (c) separating pegylated HDL particles from the product of step (b), so as to thereby prepare the composition comprising pegylated HDL particles.

In one embodiment, the HDL particles are recombinant HDL particles or mutant HDL particles. In another embodiment, the HDL particles are reconstituted HDL particles. In another embodiment, the HDL particles are native HDL particles. In yet another embodiment, the HDL particles are human HDL particles.

In one embodiment, the polyethylene glycol has a molecular weight of between 10,000 and 30,000 Daltons. In another embodiment, the polyethylene glycol has a molecular weight of between 15,000 and 25,000 Daltons. In another embodiment, the polyethylene glycol has a molecular weight of between 19,000 and 21,000 Daltons. In yet another embodiment, the polyethylene glycol has a molecular weight of about 20,000 Daltons. In an embodiment, the source of polyethylene glycol is methoxypoly(ethylene glycol).

In one embodiment, the molar ratio of polyethylene glycol to HDL particles is 2:1 to 10:1. In another embodiment, the molar ratio of polyethylene glycol to HDL particles is 4:1 to 9:1. In another embodiment, the molar ratio of polyethylene glycol to HDL particles is 7:1 to 9:1. In another embodiment, the molar ratio of polyethylene glycol to HDL particles is 7:1 to 8:1. In another embodiment, the molar ratio of polyethylene glycol to HDL particles is 8:1. In yet another embodiment, the molar ratio of polyethylene glycol to HDL particles is about 8:1.

In one embodiment, in step a) the HDL particle concentration of the mixture is 1.25-12.5 mg/ml. In another embodiment, in step (a) the HDL particle concentration of the mixture is about 5 mg/ml.

In one embodiment, in step (b) the HDL and the source of polyethylene glycol are incubated for 2-48 hours. In another embodiment, in step (b) the HDL and the source of polyethylene glycol are incubated for 4-48 hours, for 6-36 hours, for 8-30 hours, for 10-24 hours, for 12-20 hours, for 8-16 hours, for 10-14 hours or about 11 hours. In another embodiment, in step (b) the HDL and the source of polyethylene glycol are incubated for about 20 hours.

In one embodiment, in step (b) the HDL particle and the source of polyethylene glycol are incubated at 2° C.-37° C. In another embodiment, the HDL particle and the source of polyethylene glycol are incubated at 2° C.-30° C., at 2° C.-20° C., at 2° C.-10° C., or at 3° C.-6° C. In another embodiment, the HDL particle and the source of polyethylene glycol are incubated at 4° C.-6° C. In another embodiment, the HDL particle and the source of polyethylene glycol are incubated at about 4° C. In another embodiment, in step (b) the HDL and the source of polyethylene glycol are incubated at about 6° C.

In one embodiment, step (b) further comprises the step of quenching the mixture by adding about 1M glycine. In another embodiment, step (b) further comprises the step of quenching the mixture by adding glycine to a glycine concentration of 5 mM-75 mM. In another embodiment, the mixture is quenched to a glycine concentration of about 10 mM. In another embodiment, the mixture is quenched to a glycine concentration of 25-75 mM. In another embodiment, the mixture is quenched to a glycine concentration of 40-60 mM. In another embodiment, the mixture is quenched to a glycine concentration of 48-52 mM. In yet another embodiment, the mixture is quenched to a glycine concentration of about 50 mM.

In one embodiment, in step (c) the pegylated HDL particles is separated from the product of step (b) by gel-filtration chromatography. In another embodiment, the process is performed in the absence of bile salt.

The subject invention also provides a composition prepared by the processes as described herein. In one embodiment, the composition comprises monopegylated apolipoprotein A-I (ApoA-I), wherein at least 50% of the ApoA-I in the composition are monopegylated ApoA-I. In another embodiment, at least 70% or at least 80% of the ApoA-I in the composition are monopegylated ApoA-I. In another embodiment, at least 90% of the ApoA-I in the composition are monopegylated ApoA-I. In yet another embodiment, at least 98% or 99% of the ApoA-I in the composition are monopegylated ApoA-I.

In one embodiment, the composition is essentially free of multipegylated ApoA-I. In another embodiment, the composition is essentially free of bile salts.

In one embodiment, at least 50% of the total monopegylated ApoA-I in the composition are N-terminally pegylated. In another embodiment, at least 60% of the total monopegylated ApoA-I in the composition are N-terminally pegylated. In another embodiment, at least 70% of the total monopegylated ApoA-I in the composition are N-terminally pegylated. In another embodiment, at least 80% of the total monopegylated ApoA-I in the composition are N-terminally pegylated. In another embodiment, about 80% of the total monopegylated ApoA-I in the composition are N-terminally pegylated.

The subject invention also provides a method of treating a subject afflicted with an inflammatory vascular disease comprising administering to the subject an amount of the composition described herein, effective to treat the subject. In one embodiment, the inflammatory vascular disease is an atheroma or atherosclerosis. In another embodiment, the inflammatory vascular disease is atherothrombotic cardiovascular disease.

The subject invention also provides a method of treating a subject afflicted with dyslipidemia comprising administering to the subject an amount of the composition described herein, effective to treat the subject.

The subject invention also provides a method of increasing plasma HDL levels in a subject comprising administering to the subject an amount of the composition described herein, effective to increase plasma HDL levels in the subject.

The subject invention also provides a method of promoting cholesterol efflux from macrophage foam cells in a subject comprising administering to the subject an amount of the composition described herein, effective to promote cholesterol efflux from macrophage foam cells in the subject.

The subject invention also provides a method of decreasing the amount of white blood cells in a subject comprising administering to the subject an amount of the composition described herein, effective to decrease the amount of white blood cells in the subject. In an embodiment, the white blood cells include monocytes, neutrophils or both.

The subject invention also provides the compositions described herein for use in treating a subject afflicted with an inflammatory vascular disease. The subject invention also provides the compositions described herein for use in treating a subject afflicted with dyslipidemia. The subject invention also provides the compositions described herein for use in increasing plasma high-density lipoprotein levels in a subject. The subject invention also provides the compositions described herein for use in promoting cholesterol efflux from macrophage foam cells in a subject. The subject invention also provides the compositions described herein for use in decreasing the amount of white blood cells in a subject.

The subject invention also provides a method of administering a compound to a subject, the method comprising administering to the subject an amount of a composition comprising the compound bound to pegylated HDL particles, so as to thereby administer the compound to the subject. In one embodiment, the compound is lecithin cholesterol acyltransferase (LCAT). In another embodiment, the compound is wild type ApoL-1 (ApoL-1 WT).

The subject invention also provides a method of increasing the amount LCAT in a subject comprising administering to the subject an amount of a composition comprising LCAT bound to pegylated HDL particles, effective to increase the amount of LCAT in a subject.

The subject invention also provides a method of treating a subject afflicted with atherosclerosis comprising administering to the subject an amount of a composition comprising LCAT bound to pegylated HDL particles, effective to treat the subject.

The subject invention also provides a method of treating a subject afflicted with a type of a kidney disease or at risk for a type of kidney disease comprising administering to the subject an amount of a composition comprising LCAT or ApoL-1 WT bound to pegylated HDL particles, effective to treat the subject or reduce or eliminate the risk for the kidney disease in the subject.

The subject invention also provides a composition comprising a compound bound to pegylated HDL particles for use as a delivery vehicle for administering the compound to the subject.

The subject invention also provides a composition comprising the LCAT bound to pegylated HDL particles for use increasing the amount of LCAT in a subject.

The subject invention also provides a composition comprising the LCAT bound to pegylated HDL particles for use treating a subject afflicted with atherosclerosis.

The subject invention also provides a composition comprising LCAT or ApoL-1 WT bound to pegylated HDL particles for use in treating a subject afflicted with a type of a kidney disease or for reducing or eliminating the risk for the kidney disease in the subject.

For the foregoing embodiments, each embodiment disclosed herein is contemplated as being applicable to each of the other disclosed embodiments.

### DEFINITIONS

As used herein, and unless otherwise stated, each of the following terms shall have the definition set forth below.

“Administering” an agent can be effected or performed using any of the various methods and delivery systems known to those skilled in the art. The administering can be, but is not limited to, for example, intravenous, oral, intramuscular, intravascular, intra-arterial, intracoronary, intramyocardial and subcutaneous.

The term “High density lipoprotein (HDL) particle” encompasses HDL particles so referenced to by individuals skilled in the art, include pre-β HDL, β HDL, HDL₂ (including HDL₂ₐ and HDL_{2b}), HDL₃, as native or reconstituted HDL (rHDL) particles. HDL particles represent a broad group of plasma lipoproteins, which exhibit considerable diversity in their size, apolipoprotein (apo) and lipid composition. While its structural properties and compositions can be complex, in its most basic form HDL contain phospholipids and apolipoproteins (apo). More than 20 proteins have been shown to be associated with HDL particles, some of which include apolipoprotein A-I (apo A-II), apolipoprotein A-II (apo A-II), apolipoprotein A-IV (ApoA-IV), apolipoprotein A-V (Apo V), apolipoprotein C-I (Apo C-I), apolipoprotein C-II (Apo C-II), apolipoprotein C-III (Apo C-III), apolipoprotein D (Apo D), apolipoprotein E (Apo E), apolipoprotein F (Apo F), apolipoprotein M (Apo M), Lecithin cholesterol acyltransferase (LCAT), cholesteryl ester transfer protein (CETP), phospholipid transfer protein (PLTP), Paraoxonase, platelet-activating factor acylhydrolase (PAF-AH), clusterin (apo J), serum amyloid A (SAA) and tissue factor pathway inhibitor (TFPI) (von Eckardstein, 1994).

The most abundant and primary apolipoproteins is apoA-I (Ryan 2010, Assmann, 1982; von Eckardstein, 1994; Skinner, 1994; Anantharamaiah, 1991; Brouillette, 1995, Brouillette, 2001; Frank PG, 2000; Segrest, 2000, Klon, 2002), which in association with phospholipids and cholesterol, encloses a core of cholesteryl esters (Stroes, US 2008/0214434 A1). The HDL may also be mutant HDL prepared from mutant HDL apolipoproteins that have been isolated from plasma or produced by genetic engineering.

As used herein “human HDL particle” means HDL particle derived entirely or in part from human sources. The human HDL particle can be native, i.e., isolated from human serum, or reconstituted, e.g., formed by incubating human apolipoproteins, apolipoproteins fragments or peptides with phospholipid vesicles in vitro. The human HDL particle may also be recombinant human HDL particle prepared from genetically engineered human apolipoproteins or mutant human HDL prepared from mutant human HDL apolipoproteins that have been isolated from human plasma or produced by genetic engineering (Ryan 2010; Gillotte, 1996; Nissen 2003; Matsunaga 1999; Cho 2009; Zhang 2010).

A “pegylated HDL particle” is an HDL particle having a one or more PEG molecules bound thereto via a covalent bond to a protein of the HDL particle, and which is distinct from nonpegylated HDL particles. Pegylation of HDL particle pegylates the protein components but not the lipid components of the particle. Therefore, where the HDL particle, e.g., a reconstituted HDL (rHDL) particle, is such that only apolipoprotein A-I is present, then all of the pegylated proteins in the pegylated HDL particle are pegylated apolipoprotein A-I. However, where the HDL particle contain proteins other than apolipoprotein A-I, for example, the native human HDL particle, then the pegylated HDL particle may contain pegylated proteins other than pegylated apolipoprotein A-I. If the pegylation is non-selective, the ratio of the different types of pegylated proteins may be the same, assuming that the pegylation is not affected by other factors, e.g., the relative locations of the proteins. If one protein is located more towards the exterior of the HDL particle than the other, it may be expected that a higher percentage of the more exterior protein may be pegylated.

“Monopegylated”, as applied to a PEG-derivatized protein, shall mean the attachment of one, but no more than one, polyethylene glycol molecule via a covalent bond to the protein.

“Multipegylated” as applied to a PEG-derivatized protein, shall mean the attachment of more than one, for example two, three, four or more, polyethylene glycol molecules, each via a covalent bond, to the protein. For example, a protein having two polyethylene glycol molecules attached to it via a covalent bond is a multipegylated protein.

As used herein, a percent of a protein in the composition, e.g., “percent of the monopegylated ApoA-I in the composition” may refer to the percent of the monopegylated ApoA-I by weight or by molar amount. In one embodiment, the percent refers to percent by molar amount. For example, a composition comprising HDL particles which comprise ApoA-I, wherein at least 50% of the ApoA-I in the composition are monopegylated ApoA-I, means that at least 50% of the ApoA-I in the composition by molar amount are monopegylated ApoA-I.

“Essentially free of” when used in reference to multipegylated PEG-derivatized protein, e.g., multipegylated apolipoprotein A-I, shall mean, with regard to a composition, a composition wherein multipegylated apolipoprotein A-I is not present or is at a level undetectable by conventional methods. The apolipoprotein A-I (apoA-I), which is pegylated as described hereinbelow, may be wild-type apolipoprotein or a variant thereof which includes A-I Milano (Arg173Cys) or the variant (Arg173Pro), and those variants described in U.S. Pat. No. 7,223,726, issued May 29, 2007 (Oda et al.).

A “monopegylated apolipoprotein A-I” is an apolipoprotein A-I having a single PEG molecule bound thereto via a covalent bond and which is distinct from multipegylated apolipoprotein A-I, and from nonpegylated apolipoprotein A-I.

“N-terminally pegylated”, as applied to a PEG-derivatized peptide, polypeptide, or protein, shall mean the attachment of a polyethylene glycol molecule via a covalent bond to the N-terminal region of the peptide, polypeptide, or protein being derivatized, the N-terminal region being the 50%, 25% or 10% of the peptide, polypeptide, or protein nearest to the N-terminus of the molecule when in linear form. In an embodiment, N-terminally pegylated shall mean the attachment of a polyethylene glycol molecule via a covalent bond to the N-terminal residue (i.e. the N-terminus).

As used herein, an “isolated” compound is a compound isolated from a crude reaction mixture or from a natural source following an affirmative act of isolation. The act of isolation necessarily involves separating the compound from the other components of the mixture or natural source, with some impurities, unknown side products and residual amounts of the other components permitted to remain. Purification is an example of an affirmative act of isolation.

“Inflammatory vascular disease” shall mean a disease of the vascular or cardiovascular system of a human comprising an inflammatory response in a tissue of the vascular or cardiovascular system, for example a blood vessel thereof. In an embodiment, the disease is diabetic cardiovascular disease.

“Dyslipidemia” is a pathological state marked by the elevation of plasma cholesterol in a subject, triglycerides (TGs), or both, or a low high density lipoprotein level that contributes to the development of atherosclerosis. Causes may be primary (genetic) or secondary. Levels of or serum cholesterol >240 mg/dL (>6.2 mmol/L) are indicative of a dyslipidemia. Therefore, as used herein, the terms “dyslipidemia” or “dyslipidemic” refer to an abnormally elevated or decreased level of lipid in the blood plasma, including, but not limited to, the altered level of lipid associated with the following conditions: coronary heart disease; coronary artery disease; cardiovascular disease, hypertension, restenosis, vascular or perivascular diseases; dyslipidemic disorders; dyslipoproteinemia; high levels of low density lipoprotein cholesterol; high levels of very low density lipoprotein cholesterol; low levels of high density lipoproteins; high levels of lipoprotein Lp(a) cholesterol; high levels of apolipoprotein B; atherosclerosis (including treatment and prevention of atherosclerosis); hyperlipidemia; hypercholesterolemia; familial hypercholesterolemia (FH); familial combined hyperlipidemia (FCH); lipoprotein lipase deficiencies, such as hypertriglyceridemia, hypoalphalipoproteinemia, and hypercholesterolemialipoprotein.

“Essentially free of bile salts” shall mean, with regard to a composition, a composition wherein the levels of bile salts, including bile salts based on the bile acids cholic, deoxycholic, chenodeoxycholic, and lithocholic acids, are not present or are undetectable by conventional methods. Compositions that are prepared without the use of bile salts but which may nonetheless contain trace amounts of bile salts fall into this category.

The apolipoprotein A-I (apoA-I) may be wild-type apolipoprotein or a variant thereof, which includes A-I Milano (Arg173Cys) or the variant (Arg173Pro), and those variants and mimics described in, e.g., U.S. Pat. No. 7,223,726, Oda et al., issued May 29, 2007 and PCT International Application Publication No. WO 2010/093918, published Aug. 19, 2010, which are herein incorporated by reference in their entirety. The wild type human ApoA-I protein sequence is found under GenBank Accession No. NP_{—}000030, the contents of which are hereby incorporated. The apolipoprotein A-I may be that encoded by NCBI Reference Sequence: NM_{—}000039.1 or as encoded by GenBank: BC005380.1 (apolipoprotein cDNA), both sequences of which are hereby incorporated.

Apolipoprotein A-I analogs, and “monopegylated apolipoprotein A-I analogs”, are contemplated by this disclosure. Such wild-type analogs may have from 70% to 99% sequence identity to the wild-type sequence. The monopegylated apolipoprotein A-I analog is able to mediate cholesterol efflux, from e.g. human macrophage foam cells, as measured by any of the routine assays known to those in the art, including the assay described hereinbelow.

Apolipoprotein A-I purification methods are described in U.S. Pat. Nos. 6,090,921 and 6,423,830 which use an anion-exchange chromatography gel to purify ApoA, and these patents are hereby incorporated by reference in their entirety.

In an embodiment, the pegylated HDL particle is administered within the dose ranges specified by U.S. Pat. No. 7,759,315 B2, incorporated by reference. The HDL dosage ranges are from 0.1-200 mg. In another embodiment, the dosage ranges are from 20 mg apoA-I/kg body weight to 200 mg apoA-I/kg body weight per treatment. In another embodiment, the dosage ranges are from 10-80 mg, HDL (weight based on apolipoprotein) per kg body weight per treatment. For example, the dosage of HDL which is administered may be about 0.2-100 mg HDL per kg body weight (weight based on apolipoprotein) given as an intravenous injection and/or as an infusion for a clinically necessary period of time, e.g. for a period ranging from a few minutes to several hours, e.g. up to 24 hours. If necessary, the HDL administration may be repeated one or several times. The actual amount administered will be determined both by the nature of the disease which is being treated and by the rate at which the HDL is being administered. Doses can be administered of differing amounts during treatment, for example one or two high bolus doses at the beginning of treatment followed by lower maintenance doses.

Production of reconstituted HDL is described, by way of example in U.S. Pat. No. 5,652,339. Production of recombinant HDL is described, by way of example in U.S. Pat. No. 6,559,284, and PCT International Patent Application Publications WO 87/02062 (in E. coli, yeast and CHO cells). The contents of these documents and U.S. Pat. No. 7,759,315 B2 are incorporated herein by reference.

For the therapeutic or prophylactic use of HDL particles in humans, a dose of HDL in gram quantities is necessary to achieve significant increases of the apoA-I or HDL particle level in the plasma. (See, e.g. U.S. Pat. No. 5,652,339)

In an embodiment, the pharmaceutical formulation or the pegylated HDL particle is administered to a subject once weekly for about 6 months, about 5 months, about 4 months, about 3 months, about 2 months or about 1 month. In an embodiment, the pharmaceutical formulation or the pegylated HDL particle is administered about every day, about every other day, about every 3 days, about every 4 days, about every 5 days, about every 6 days, about every 7 days, about every 8-10 days or about every 11-14 days.

In an embodiment the pegylated HDL particle is administered as a liquid pharmaceutical formulation having an osmolality of about 280 to about 320 mOsm. In an embodiment the pegylated HDL particle is administered as a liquid pharmaceutical formulation having an osmolality of about 290 mOsm.

One method of delivery of monopegylated ApoA-I (wild type or mutant protein) embedded in the HDL particle is through intravenous infusion of large quantities of protein to patients as described by Chiesa G 2002 and Sirtori 1999. The monopegylated apoA-I (wild type or mutant protein) embedded in the HDL particle can be administered singly or in combination with other cardiovascular or triglyceride-lowering drugs, for example statins. They may be conventionally prepared with excipients and stabilizers in sterilized, lyophilized powdered forms for injection, or prepared with stabilizers and peptidase inhibitors of oral and gastrointestinal metabolism for oral administration. They may also be administered by methods including, but not limited to, intravenous, infusion, or intramuscular administration. In one embodiment, the pegylated HDL particle is administered by intravenous infusion into a peripheral vein of a subject. In embodiments the pegylated HDL particle (whether alone or in a pharmaceutical composition) is administered intravenously into the fossa of the arm or a central line into the chest. In embodiments, the pharmaceutical formulation is infused into the cephalic or median cubital vessel at the antecubital fossa in the arm of a subject.

As used herein, a “pharmaceutical carrier” is a pharmaceutically acceptable solvent, suspending agent or vehicle, for delivering the instant compounds to the animal or human. The carrier may be liquid, aerosol, gel or solid and is selected with the planned manner of administration in mind. In an embodiment, the pharmaceutical carrier is a sterile pharmaceutically acceptable solvent suitable for intravenous administration.

In an embodiment the sterile liquid pharmaceutical formulation comprises a sucrose-mannitol carrier and a phosphate buffer. In an embodiment the sucrose-mannitol carrier comprises about 6.0% to about 6.4% sucrose and about 0.8% to about 1% mannitol. In an embodiment the sucrose-mannitol carrier comprises about 6.2% sucrose and about 0.9% mannitol. In an embodiment the sterile liquid pharmaceutical formulation has a pH of about 7.0 to about 7.8. In an embodiment the sterile liquid pharmaceutical formulation has a pH is about 7.5. Formulations and methods of administration of HDL particles with apolipoprotein A-I to subjects are described in U.S. Pat. No. 7,435,717, the contents of which are hereby incorporated in its entirety, and which formulations and methods may be used for the pegylated HDL particles with apolipoprotein A-I described herein.

Other injectable drug delivery systems include solutions, suspensions, and gels. Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

In an embodiment, the subject being treated has a HDL-cholesterol level of below 45 mg/dl for a man or below 50 mg/dl for a woman. In an embodiment the subject being treated (male or female) has a HDL-cholesterol of <40 mg/dL [<1.04 mmol/L].

The pegylated HDL and compositions in the methods of treatment described herein are used in effective amounts. As used herein, the term “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

As used herein, “treatment of the diseases” or “treating” a disease, disorder or condition, e.g. of a cardiovascular disease, encompasses inducing inhibition, regression, or stasis of the diseases, disorder or condition, or ameliorating or alleviating a symptom of the disease, disorder or condition. In an embodiment, treatment of diseases having atheroma as a symptom thereof comprises reduction in the volume of the atheroma. Non-limiting examples include atheroma and atherosclerosis.

As used herein, “a subject afflicted with” a disease, condition or disorder means a subject who was been affirmatively diagnosed to have the disease, condition or disorder.

As used herein, “polyethylene glycol” (PEG), unless otherwise stated, means, in regard to a pegylated molecule, a polymer having the formula OH—CH₂—(CH₂—O—CH₂)ₙ—CH₂—OH, wherein one of the hydroxy groups is replaced by a covalent bond to the molecule being pegylated and wherein n is the number of oxyethylene groups. The polyethylene glycol useful herein can have an average molecular weight of 5,000 to 40,000. The actual molecular weight of each PEG molecule should be no less than 90% of and no greater than 110% of the average molecular weight. In specific embodiments the average molecular weight of the PEG is 5,000, 10,000, 20,000, 30,000 and 40,000. In an embodiment, the PEG is a branched PEG. In embodiments the PEG can be straight chain, substituted or unsubstituted.

As used herein, “source of polyethylene glycol” shall mean any art-recognized source of PEG for purposes of pegylating a molecule. Non-limiting examples include methoxy PEG (e.g. in the form of methoxy PEG propionaldehyde, of molecular weight 20,000 available from JenKem Technology USA, Allen, Tex.). Other sources include Polyethylene glycol 200, 300, 400, 600, 1000, 1450, 3350, 4000, 6000, 8000, 20000, 30000 and 40000 (CAS No.: 25322-68-3), and tresyl monomethoxy PEG (TMPEG). Branched and multiarm PEG may also be used. Pegylation techniques are discussed in Roberts et al., Adv Drug Deliv Rev. 2002 Jun. 17; 54(4):459-76, hereby incorporated by reference in its entirety. Activated PEGs that target —NH₂, —OH or —SH can be used.

In an embodiment, the Pegylated molecule is made as follows: PEG aldehyde was stored at −20° C. and taken from storage and fully equilibrated to room temperature before use. Human HDL particles were reconstituted in 50 mM sodium acetate, pH 5.5, 10 mM sodium cyanoborahydride at concentration of ˜4 mg/ml. The amount of PEG aldehyde to be used (molar ratio PEG:HDL protein=8:1) was calculated and dissolved in the buffer used 50 mM sodium acetate, pH 5.5, 10 mM sodium cyanoborahydride. Then, PEG solution was added slowly to protein solution with gentle swirling. The mixture was incubated at 4° C. overnight. The reaction was quenched with addition of glycine to 50 mM and incubated for 16 hours at 4° C.

As used herein, “about” with regard to a stated number encompasses a range of ±1% of the stated value. By way of example, about 100 mg/kg therefore includes 99, 99.1, 99.2, 99.3, 99.4, 99.5, 99.6, 99.7, 99.8, 99.9, 100, 100.1, 100.2, 100.3, 100.4, 100.5, 100.6, 100.7, 100.8, 100.9 and 101 mg/kg. Accordingly, about 100 mg/kg includes, in an embodiment, 100 mg/kg.

As used herein, an “amount” or “dose” of a compound, e.g., measured in milligrams refers to the weight of the compound present in a drug product, regardless of the form of the drug product.

As used herein, “inhibition” of disease progression or disease complication in a subject means preventing or reducing the disease progression and/or disease complication in the subject.

It is understood that where a range is provided, all integers within that range, and tenths thereof, are also provided by the invention. For example, “0.2-5 mg/kg” is a disclosure of 0.2 mg/kg, 0.3 mg/kg, 0.4 mg/kg, 0.5 mg/kg, 0.6 mg/kg etc. up to 5.0 mg/kg.

The treatment with the pegylated HDL may be a component of a combination therapy or an adjunct therapy, i.e. the subject or patient in need of the drug is treated or given another drug for the disease in conjunction with one or more of the instant compounds, e.g. statins, niacin, estrogen, ezemtimibe, nicotinic acid. This combination therapy can be sequential therapy where the patient is treated first with one drug and then the other or the two drugs are given simultaneously or contemporaneously. These can be administered independently by the same route or by two or more different routes of administration depending on the dosage forms employed.

All combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Pegylation of Human apoA-I

Pegylated form of human apoA-I retains its key biological activity, i.e. promotion of cholesterol efflux from macrophage foam cells.

Methoxy PEG Propionaldehyde (M-PEG-ALD), MW 20000 was purchased from JenKem Technology USA (Allen, Tex.). The PEG-containing bottle was removed from storage and fully equilibrated to room temperature. ApoA-I reconstituted in 50 mM sodium acetate, pH 5.5, 10 mM sodium cyanoborahydride was dissolved. PEG Aldehyde was used at a molar ratio of 10:1, PEG:apoA-I. The required amount of PEG was dissolved in an aliquot of 50 mM sodium acetate, pH 5.5, 10 mM sodium cyanoborahydride. The PEG preparation was mixed with the human apoA-I solution with gentle swirling. The final concentration of human apoA-I was about 2 to 6 mg/ml. The mixture was incubated at 4° C. overnight. To minimize the formation of multipegylated species, the reaction was quenched by adding IM glycine to a concentration of 10 mM or to a concentration of 50 mM. Under these conditions, apoA-I was preferentially pegylated at the N-terminus. The pegylated human apoA-I was analyzed by SDS-PAGE and Coomassie Brilliant Blue staining and shown in FIG. 1.

After ˜16 hours incubation at 4° C., approximately half of human apoA-1 molecules were pegylated (FIG. 1A). The pegylated apoA-I appeared to be largely a homologous species under these conditions. Even though a monopegylated species was observed, the reaction was incomplete as there was still significant native apoA-I in the preparation (FIG. 1A-2). If incubation temperature was changed to room temperature, a large portion of apoA-I was pegylated (FIG. 1B). However, multipegylation of apoA-I appeared to be dominant under these conditions because multiple species of PEG-apoA-I were generated with higher molecular weight (FIG. 1B)

### Example 2

### Cholesterol Efflux from Macrophage Cells

Mouse peritoneal macrophages were loaded with cholesterol by incubating the cells with a modified LDL preparation (100 μg acetyl-LDL protein/ml) in the presence of radioactive cholesterol tracer ([³H] cholesterol). The cells were washed three times with the last wash after equilibrium in cell culture media plus 0.2% bovine albumin for 30 minutes. Cholesterol efflux was then initiated by addition of unmodified native apoA-I or pegylated apoA-I preparation. In order to rule out the potential effect of free PEG molecules in the efflux assay, the same amount of quenched PEG preparation was added to the group of unmodified apoA-I preparation during the efflux assay. The results are shown in FIG. 2.

As shown, pegylation of human apoA-I did not adversely affect the activity of apoA-I to promote cholesterol efflux. Cholesterol efflux from macrophage foam cells to apoA-I or PEG-apoA-I showed a similar dose response (FIG. 2). The PEG-apoA-I preparation used in the efflux assays in FIG. 2 was from the preparations as shown in FIG. 1A. Multipegylation of apoA-I as shown in FIG. 1B, however, caused decrease of cholesterol efflux from macrophage foam cells (FIG. 2). Therefore, the multipegylated apoA-I has loss of function as shown by reduced capacity to promote cholesterol efflux from macrophage foam cells (FIG. 2B).

### Example 3

### Pegylation of Human HDL

### Methods

1) Isolation of HDL particles. Human HDL was isolated by density gradient centrifugation from human plasma. Non-HDL lipoproteins were first removed from the plasma by density gradient centrifugation at d=1.063. Then HDL was collected as the top lipoprotein layer of the density gradient centrifugation at d=1.21.

2) Pegylation of HDL. Procedure Methoxy PEG Propionaldehyde (M-PEG-ALD), MW 20000 was purchased from JenKem Technology USA (Allen, Tex.). Fully equilibrate PEG to room temperature. Human HDL was isolated by density gradient centrifugation from human plasma. Non-HDL lipoproteins were first removed from the plasma by density gradient centrifugation at d=1.063. Then HDL was collected as the top lipoprotein layer of the density gradient centrifugation at d=1.21. Purified human HDL was reconstituted in 50 mM sodium acetate, pH 5.5, 10 mM sodium cyanoborahydride. The amount of PEG Aldehyde to be used was calculated as molar ratio=8:1, PEG: HDL protein. The required amount of PEG is dissolved in an aliquot of 50 mM sodium acetate, pH 5.5, 10 mM sodium cyanoborahydride and mixed the PEG preparation with the human HDL solution with gentle swirl. The final concentration of human HDL protein was ˜5 mg/ml. The mixture was incubated at 4° C. overnight. To minimize the formation of multipegylated species, the reaction was quenched by adding 1M glycine to a concentration of 50 mM. Under these conditions, HDL proteins were preferentially pegylated at the N-terminus.

3) Isolation of pegylated HDL particles. Pegylated HDL can be readily separated and purified from the unincorporated PEG molecules by gel-filtration chromatography.

4) Coomassie Blue Staining: Gel was prefixed in 50% MeOH, 10% HoAC, 40% H2O for 30 minutes. Stain gel in the above solution, with 0.25% Coomassie Brilliant Blue R-250, for 2-4 hours, until the gel was a uniform blue color. Staining was complete when the gel was no longer visible in the dye solution. Destain for 4-24 hours in 5% MeOH, 7.5% HoAC, 87.5% H2O with multiple changes of the same solution. Destain until background was clear.

5) Western blot: Mix ˜5 to 200 μg HDL protein 1:1 (v:v) with Laemmli buffer and heat to 80° C. for 5 min. The samples were loaded onto SDS-PAGE gel and the gel was run until the blue front is at the bottom of the gel. Transfer proteins from the gel onto nitrocellulose membranes for immunoblotting analysis. Block the membrane for 30 min in 20-30 ml 1× Tris buffer supplemented with 5% non-fat dry milk and 0.1% Tween 20, on a shaker. Incubate with primary anti-human apoA-I antibody diluted Tris buffer, 5% milk and 0.1% Tween 20. Incubate at room temperature for 4 hrs. Wash 3 times for 5-10 min in ˜50 ml 1× Tris buffer with 0.1% Tween 20 at room temperature on a shaker. Incubate with secondary antibody for 30 min to 1 hr at room temperature. Amersham HRP-conjugated anti-rabbit antibody was used as the secondary antibody. Wash 3 times, 10 min each in about 50 ml 1× Tris buffer with 0.1% Tween 20 at room temperature. Detect protein with ECL kit from PIERCE (IL). In a separate tube, mix black and white ECL solutions in a 1:1 ratio. Aliquot solution onto membranes and allow reaction to proceed for 1 minute. Drain the ECL, wrap in plastic and expose to film.

6) Macrophage cholesterol efflux: Mouse macrophage-like RAW cells were cultured in DMEM medium plus 10% fetal bovine serum. The cells were labeled with [³H]cholesterol (0.5 Ci/ml) overnight. The cells were washed with DMEM plus 0.2% bovine serum albumin three times and the efflux was initiated by addition of DMEM medium, 0.2% bovine serum albumin in the presence or absence of indicated amount of HDL or apoA-I. The efflux proceeded for 2 to 8 hours. Then the medium was collected. The cells were lysed with lysis buffer (PBS plus 0.1 N NaOH and 0.1% SDS). The radioactivity of the medium and cell lysates was determined using a beta liquid scintillation counter.

7) Half life experiment: Native or PEG-HDL at the indicated amount in a volume of 100-300 μl was injected into 3-5 month old C57BL6/J mice (Jackson Laboratory, Main) via the tail vein. At the indicated time point, an aliquot of blood was collected from the mice and plasma was isolated by centrifugation. Then an aliquot of the plasma equivalent to 0.05 to 0.2 μl of plasma was subjected to SDS-PAGE and Western analysis as described above. The density of the protein band on the developed film was determined by densitometry. The half life of the native or PEG-apoA-I in vivo was estimated by the clearance of the human apolipoprotein in blood circulation.

8) The source of anti-human apoA-I antibody is from BINDING SITE, Birmingham, UK., Cat. # PC085.

### Results

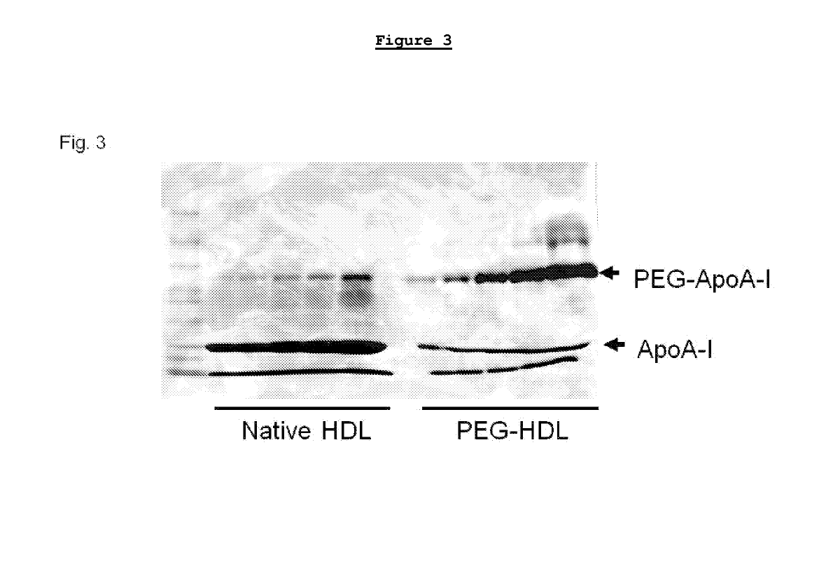

The pegylated human HDL was analyzed by SDS-PAGE and Coomassie Brilliant Blue staining and shown in FIG. 3. After ˜16 hours incubation at 4° C., >90% of human apoA-I in HDL particles was pegylated (FIG. 3, the pegylated apoA-I in HDL is indicated as PEG-ApoA-I and the unmodified native apoA-I in HDL is indicated as ApoA-I). The pegylated HDL particle was largely a homogeneous species under these conditions.

### Example 3A

### Pegylated Human HDL is Biologically Active to Promote Cholesterol Efflux from Macrophage Foam Cells

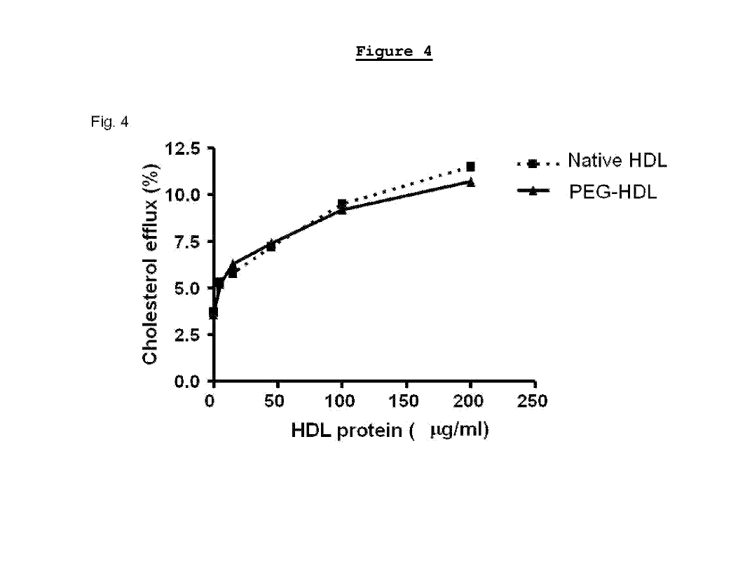

A major mechanism proposed for the anti-atherogenic property of HDL is reverse cholesterol transport, in which HDL transports cholesterol from the peripheral tissue such as macrophage foam cells back to the liver for disposal. In this scenario, cholesterol efflux from macrophage foam cells to HDL constitutes the initial step of reverse cholesterol transport. It has been well documented that HDL acts as cholesterol acceptors and promotes cholesterol efflux from macrophage foam cells. Therefore, it is important to test the biological activity of pegylated HDL to promote cholesterol efflux. In this experiment the N-terminally pegylated human HDL particles were tested for their ability to promote cholesterol efflux from macrophages as compared to native HDL. Murine macrophage-like RAW cells are labeled with radioactive cholesterol tracer ([³H]cholesterol). After washing cells with the culture media to remove the extracellular cholesterol tracer, cholesterol efflux was initiated by addition of unmodified native human HDL or pegylated HDL as indicated in FIG. 4. In order to rule out the potential effect of free PEG molecules in the efflux assay, the same amount of quenched PEG preparation was added to the group of unmodified HDL preparation during the efflux assay. The results are shown in FIG. 4. The targeted pegylation of human HDL particles did not apparently affect the activity of HDL to promote cholesterol efflux. Cholesterol efflux from macrophages to the native HDL or PEG-HDL showed a similar dose response (FIG. 4). The PEG-HDL preparation used in the efflux assays in FIG. 4 was from the preparations as shown in FIG. 3.

### Example 3B

### Pegylated Human HDL Particles have an Increased Half Life In Vivo

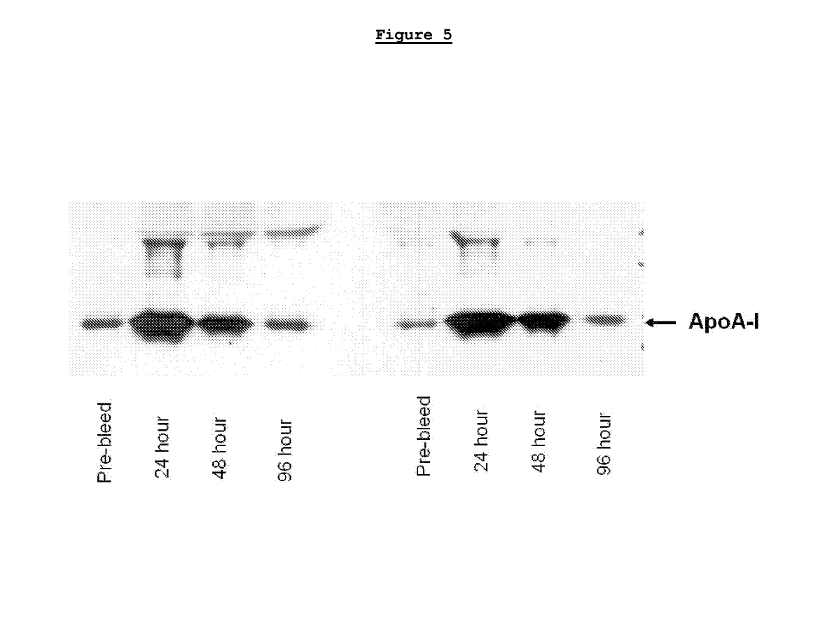

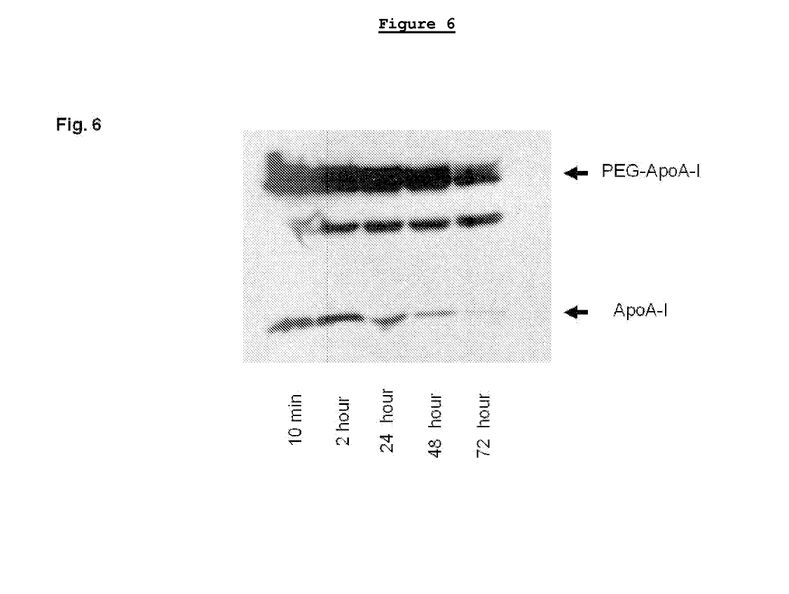

In order to evaluate the pharmacokinetics and clearance of PEG-HDL particles in blood circulation, the turnover of unpegylated HDL is compared with that of the PEG-HDL particles in vivo in mice. FIG. 5 shows the turnover of 0.8 mg unpegylated HDL injected into mouse circulation via the tail vein. The half life of HDL protein (mainly apoA-I) estimated by Western analysis of an aliquot of plasma is approximately 7 hours. At 96 hours after injection, all human apoA-I appeared to be cleared (the remaining signal appears to be the endogenous mouse apoA-I, as the density of the band is similar to that of the pre-bleed control). N-terminally pegylated human HDL (20 kDa M-PEG-ALD was used in this case) was then injected into mice. FIG. 6 shows the turnover of ˜1 mg PEG-HDL particles in mouse plasma. The turnover time of PEG-HDL, estimated by the clearance of PEG-apoA-I in HDL, was clearly increased as compared with that of unpegylated human HDL (FIG. 6 vs FIG. 5). The estimated half life is ˜24 hours i.e., the pegylated HDL particles showed a 3-fold increase in half-life compared to unpegylated HDL particles. There was still a considerable amount of PEG-HDL remaining in the plasma after 72 hours.

### Example 4

### Pegylated Human HDL Particles and Inflammatory Vascular Diseases

Subjects having an inflammatory vascular disease are intravenously administered an amount of a composition having pegylated HDL particles described hereinabove. The administration can be over a period of 1-2 months at a dose of 15 mg/kg to 45 mg/kg of mg apoA-I/kg body weight. Treatment progress can be measured by standard ultrasound methods. The pegylated HDL particle can be administered in a sterile liquid pharmaceutical formulation comprising a sucrose-mannitol carrier and a phosphate buffer. The sucrose-mannitol carrier can comprise about 6.2% sucrose and about 0.9% mannitol. The sterile liquid pharmaceutical formulation can have a pH of about 7.5. The liquid pharmaceutical formulation can have an osmolality of about 290 mOsm. The pegylated HDL particle can be administered as an HDL particle liquid pharmaceutical formulation comprising less than 6,000 particulates greater than 10 μm in size per 50 mL or less than 600 particulates greater than 25 μm in size per 50 mL.

In cases where the inflammatory vascular disease is atheroma, a reduction in atheroma volume is observed. In cases where the inflammatory vascular disease is atherosclerosis, a decrease in plaque size is observed.

Subjects having a dyslipidemia are intravenously administered a composition comprising high density lipoprotein (HDL) particles described hereinabove over a period of one month to two months. The administration of the pegylated HDL composition is found to ameliorate the dyslipidemia in the subjects.

Intravenous administration of a composition comprising HDL particles which comprises pegylated apolipoprotein A-I described hereinabove over a period of 1-2 months increases plasma HDL levels in subjects having a HDL-cholesterol level of below 45 mg/dl for a man or below 50 mg/dl for a woman. Intravenous administration of a composition comprising HDL particles which comprises pegylated apolipoprotein A-I described hereinabove over a period of 1-2 months also increases plasma HDL levels in subjects having a HDL-cholesterol level of below 40 mg/dl for a man. The pegylated HDL particle composition is administered as an HDL particle liquid pharmaceutical formulation comprising less than 6,000 particulates greater than 10 μm n size per 50 mL or less than 600 particulates greater than 25 μm in size per 50 mL.

### Discussion

While pharmacologic intervention to treat atherosclerosis traditionally focused on lowering LDL-cholesterol levels as a therapeutic target, a number of intervention trials have also shown that there is an inverse correlation between the plasma concentration of HDLs and the incidence of cardiovascular disease. The epidemiologic evidence in support of a cardioprotective function for HDL-cholesterol suggests that HDLs influence the cellular processes involved in atherogenesis. Therefore, high-density lipoprotein (HDL) therapy has drawn increased attention from researchers, and has shown great potential to become a new approach that is complementary to existing therapies for atherosclerosis. However, one problem concerning the direct infusion of HDL, recombinant apo-AI or apo-AI-phospholipid complexes is achieving and maintaining plasma HDL at its sufficient therapy concentration over an appropriate period of time without causing adverse effects in humans. In addition, making suitable HDL particle compositions without bile salts has not been reported.

Disclosed here is a method of pegylating human HDL with activated methoxypoly(ethylene glycol) (MPEG) to increase the plasma half life of human HDL. Importantly, the pegylated form of human HDL made still retains its biological activities such as promotion of cholesterol efflux from macrophage foam cells. Additionally, pegylated HDL turnover was assessed by injecting either the unpegylated HDL particles or pegylated HDL particles into the tail vein of mice. The results revealed that the estimated half life is approximately 3-fold increased compared to unpegylated HDL particles.

Another advantage of this technology is that the purification of pegylated HDL product is simple and efficient. Pegylated HDL product can be quickly purified by gel-filtration chromatography.

PCT Publication No. WO 2010/141097 disclosed that monopegylated purified apoA-I has an increased half life, cholesterol promoting activity and could incorporate into reconstituted HDL particles. However, multipegylated purified apoA-I proved to have qualities the opposite of that desired. The multipegylated form decreased cholesterol efflux from macrophages when tested. Thus the monopegylated form of human apoA-I is a more efficient form of human apoA-I for therapy for cardiovascular and/or inflammatory diseases, since monopegylated apoA-I has a significantly higher half life and maintains its biological activity. However, the efficiency in generating monopegylated apoA-I was an obstacle, with no more than 40-50% monopegylated apoA-I being produced by the process of PCT Publication No. 2010/141097.

Thus, pegylating the HDL particle itself, by monopegylating one or more proteins of the HDL particle, is an approach which provides surprisingly positive results. Experiments conducted using the method described herein found that approximately 90% of the protein (which is predominantly apoA-I) affiliated with HDL particles became monopegylated. Additionally, there was no significant multipegylation of apoA-I affiliated with the HDL particle. It is hypothesized that, because apoA-I was embedded within the HDL particle, the N-terminus of apoA-I was available for monopegylation. Thus, not only was the pegylation process more specific, but the process herein was more efficient.

In addition, this method of producing the HDL particles does not require the use of bile salts. This is an important feature, as bile salts can cause adverse health effects in humans.

Overall, without wishing to be bound to any particular theory, these studies suggest the following: N-terminal pegylation using human HDL or reconstituted HDL as the substrate increases the efficiency of targeted pegylation of human HDL protein, particularly HDL apoA-I. The pegylated human HDL promotes cholesterol efflux from macrophages as potently as the native HDL, with an increased half life in vivo. Therefore, targeted pegylation of human HDL or apoA-I/phospholipid complexes in rHDL as the substrate represents a superior method to increase the efficacy of human apoA-I- or HDL-mediated therapy for cardiovascular or inflammatory diseases.

### Example 5

### Examination of Effect of rHDL Vs. PED-rHDL on Atherogenesis In Vivo

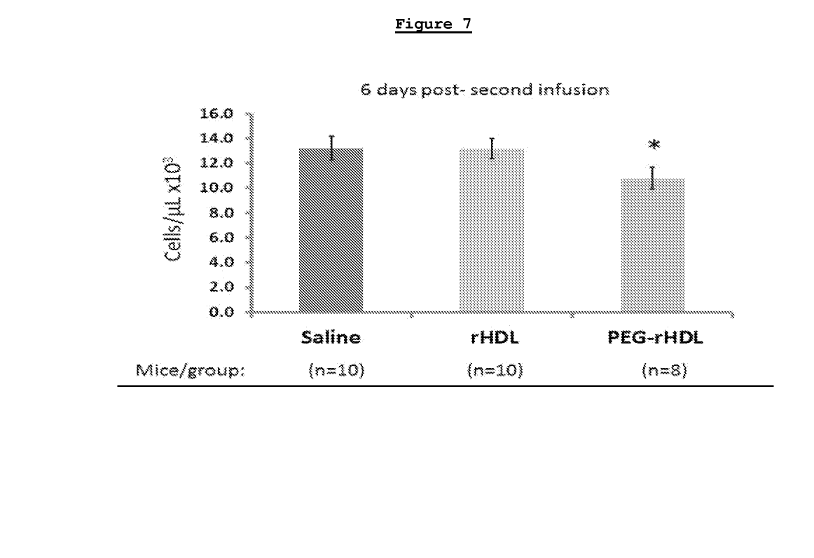

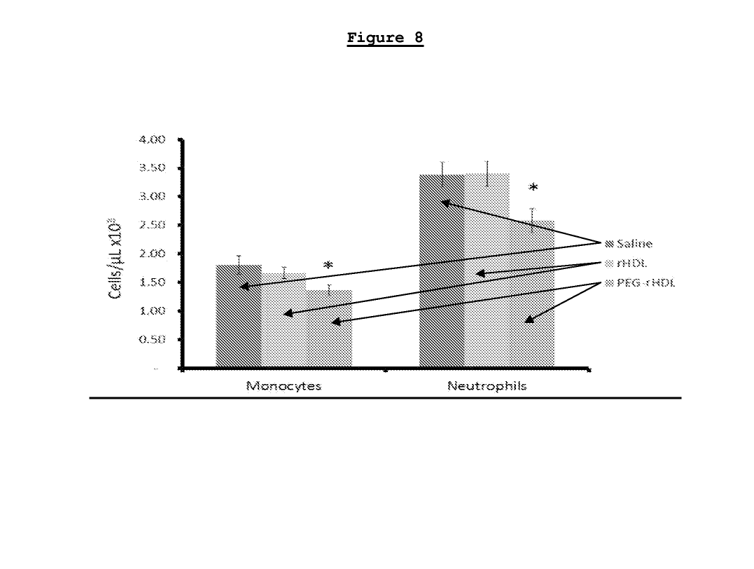

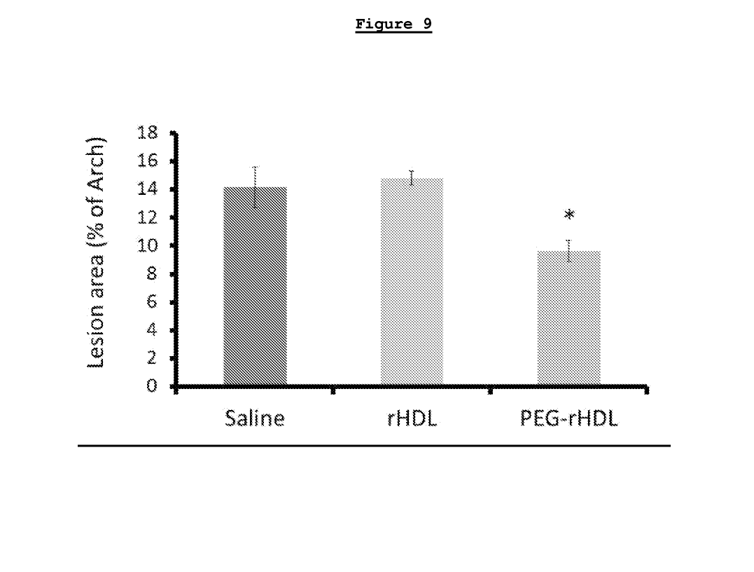

ApoE^{-/-} mice fed the high fat high cholesterol diet for 9 weeks were given over three week period two injections of saline, rHDL or PEG-rHDL, at dose of 40 mg/ml HDL protein, which showed marginal effects of rHDL on peripheral monocyte and neutrophil count in apoE^{-/-} mice in the previous studies. Then peripheral while blood cell profiles and atherogenesis were determined. As compared with the saline-treated mice, rHDL infusion showed no significant effects on peripheral total white blood cell, monocyte or neutrophil count (FIGS. 7 and 8) nor did it have any effect on en face aortic atherosclerotic lesion area (FIG. 9). Strikingly, PEG-rHDL at the same dose significantly decreased total white blood cell, monocyte and neutrophil count (FIGS. 7 and 8) as well as the atherosclerotic lesion area (FIG. 9). These results indicate that PEG-rHDL is more potent to reduce atherosclerosis than rHDL, consistent with the prolonged half life of PEG-rHDL in vivo.

This study showed that 1) PEG-HDL is more effective than HDL at reducing atherosclerosis; 2) PEG-HDL can be used at a lower dose than HDL, with decreased side effects and increased therapeutic efficacy; 3) this can translate into lower cost of production, a major factor in rHDL preparation.

### Example 6

### Cholesterol Efflux from Macrophage Foam Cells

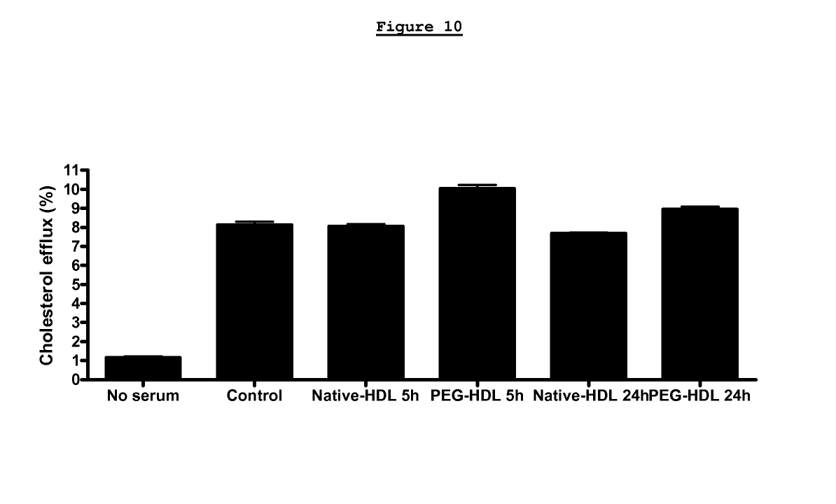

An experiment was conducted where 1 mg human PEG-HDL was injected into mice and compared with 1 mg control HDL. Then a macrophage cholesterol efflux assay was taken at 5 hours and 24 hours from plasma samples. It was found that PEG-HDL made according to the method described herein had superior ability to increase serum cholesterol efflux capacity. (Results shown in FIG. 10) The control serum and Native-HDL samples resulted in a similar level of cholesterol efflux, This observation may be in part explained by the fact that the control serum contained residual cholesterol (and perhaps HDL).

### Example 7

### Pegylated HDL Particles Treat Inflammatory Vascular Diseases

The complexes and compositions can be used for prophylactic or therapeutic treatment of a number of diseases including but not limited to cardiovascular disease (e.g., acute coronary syndrome (ACS, atherosclerosis and myocardial infarction) or diseases, disorders or conditions such as diabetes, stroke or myocardial infarction that predispose to ACS, hypercholesterolaemia (e.g., elevated serum cholesterol or elevated LDL cholesterol) and hypocholesterolaemia resulting from reduced levels of high-density lipoprotein (HDL), such as is symptomatic of Tangier disease.

The complexes and compositions can be used to treat or prevent dyslipidemia and/or virtually any disease, condition and/or disorder associated with dyslipidemia. Diseases associated with dyslipidemia include, but are not limited to coronary heart disease, coronary artery disease, acute coronary syndrome, cardiovascular disease, hypertension, restenosis, vascular or perivascular diseases; dyslipidemic disorders; dyslipoproteinemia; high levels of low density lipoprotein cholesterol; high levels of very low density lipoprotein cholesterol; low levels of high density lipoproteins; high levels of lipoprotein Lp(a) cholesterol; high levels of apolipoprotein B; atherosclerosis (including treatment and prevention of atherosclerosis); hyperlipidemia; hypercholesterolemia; familial hypercholesterolemia (FH); familial combined hyperlipidemia (FCH); lipoprotein lipase, deficiencies, such as hypertriglyceridemia, hypoalphalipoproteinemia, and hypercholesterolemialipoprotein.

A composition comprising pegylated HDL particles as described herein is administered to a subject afflicted with an inflammatory vascular disease. The administration of the composition is effective to treat the subject.

A composition comprising pegylated HDL particles as described herein is administered to a subject afflicted with an atheroma. The administration of the composition is effective to treat the subject.

A composition comprising pegylated HDL particles as described herein is administered to a subject afflicted with atherosclerosis. The administration of the composition is effective to treat the subject.

A composition comprising pegylated HDL particles as described herein is administered to a subject afflicted with atherothrombotic cardiovascular disease. The administration of the composition is effective to treat the subject.

A composition comprising pegylated HDL particles as described herein is administered to a subject afflicted with dyslipidemia. The administration of the composition is effective to treat the subject.

A composition comprising pegylated HDL particles as described herein is administered to a subject. The administration of the composition is effective to increase plasma high-density lipoprotein levels in the subject.

A composition comprising pegylated HDL particles as described herein is administered to a subject. The administration of the composition is effective to promote cholesterol efflux from macrophage foam cells in the subject.

A composition comprising pegylated HDL particles as described herein is administered to a subject. The administration of the composition is effective to decrease the amount of white blood cells in the subject.

A composition comprising pegylated HDL particles as described herein is administered to a subject. The administration of the composition is effective to decrease atheroscleotic lesions in the subject.

### Example 8

### Pegylated HDL Particles as Delivery Vehicles for Molecules Bound to HDL

Pegylated HDL particles can be used as nanoparticles for delivering HDL bound materials into the body. For example, the lecithin cholesterol acyltransferase (LCAT) can be delivered on PEG-HDL and benefit from the extended half-life. This can be a treatment for human LCAT deficiency to prevent kidney disease, or for atherosclerosis. In addition, the wild type ApoL-1 (ApoL1-WT) can be delivered by being bound to PEG-HDL to subjects with the risk variant of ApoL1 for different kinds of kidney diseases.

Pegylated HDL particles as described herein are bound to LCAT enzymes. The enzymes bound to the pegylated HDL particle are administered to subjects afflicted with lecithin cholesterol LCAT deficiency and the administration of the enzymes bound to pegylated HDL particles is effective to increase the level of LCAT in the subject or to treat the subject. In addition, the enzymes bound to pegylated HDL particles have prolonged half life in the body as compared to the enzymes which are not bound to pegylated HDL particles, or enzymes which are bound to non-pegylated HDL particles.

Pegylated HDL particles as described herein are bound to LCAT enzymes. The enzymes bound to the pegylated HDL particle are administered to subjects afflicted with a type of kidney disease or at risk for a type of kidney disease. The administration of the enzymes bound to pegylated HDL particles is effective to treat the subject, reduce the risk of kidney disease, or prevent kidney disease in the subject. In addition, the enzymes bound to pegylated HDL particles have prolonged half life in the body as compared to the enzymes which are not bound to pegylated HDL particles, or enzymes which are bound to non-pegylated HDL particles.

Pegylated HDL particles as described herein are bound to LCAT enzymes. The enzymes bound to the pegylated HDL particle are administered to subjects afflicted with atherosclerosis and the administration of the enzymes bound to pegylated HDL particles is effective to treat the subject. In addition, the enzymes bound to pegylated HDL particles have prolonged half life in the body as compared to the enzymes which are not bound to pegylated HDL particles, or enzymes which are bound to non-pegylated HDL particles.

Pegylated HDL particles as described herein are bound to wild type ApoL-1 (ApoL1-WT). The ApoL1-WT bound to pegylated HDL particles are administered to subjects afflicted with a type of kidney disease associated with risk allelic variants of apoL-1 and the administration of the ApoL1-WT bound to pegylated HDL particles is effective to treat the subject or reduce or eliminate risk for the kidney disease in the subject. In addition, the ApoL1-WT bound to pegylated HDL particles have prolonged half life in the body as compared to ApoL1-WT which are not bound to pegylated HDL particles, or ApoL1-WT which are bound to non-pegylated HDL particles.

Pegylated HDL particles as described herein are bound to ApoL1-WT. The ApoL1-WT bound to pegylated HDL particles are administered to subjects with allelic variants of ApoL1 associated with risk of a type of kidney disease, and the administration of the ApoL1-WT bound to pegylated HDL particles is effective to reduce or eliminate the risk of kidney disease in the subject. In addition, the ApoL1-WT bound to pegylated HDL particles have prolonged half life in the body as compared to ApoL1-WT which are not bound to pegylated HDL particles, or ApoL1-WT which are bound to non-pegylated HDL particles.

### REFERENCES

1. Anantharamaiah G M, Brouillette C G, Engler J A et al. “Role of amphipathic helixes in HDL structure/function.” Adv Exp Med Biol 1991; 285:131-140.
2. Assmann G. 1982 “Lipid Metabolism and Atherosclerosis.” New York: Schattauer.
3. Barter P J, Brewer H B Jr, Chapman M J et al. “Cholesteryl ester transfer protein: a novel target for raising HDL and inhibiting atherosclerosis.” Arterioscler Thromb Vasc Biol 2003; 23:160-167.
4. Brouillette C G, Anantharamaiah G M, Engler J A et al. “Structural models of human apolipoprotein A-I: a critical analysis and review.” Biochim Biophys Acta 2001; 1531:4-46.
5. Brouillette C G, Anantharamaiah G M. “Structural models of human apolipoprotein A-I.” Biochim Biophys Acta 1995; 1256:103-129.
6. Chiesa G. and Sirtori C R, (2002) “Use of recombinant apolipoproteins in vascular diseases: the case of apoA-I” Curr Opin Investig Drugs March 2002; 3(3):420.
7. Cho (2009) “A reconstituted high density lipoprotein containing the V156E mutant of apolipoproteins A-I exhibits anti-atherosclerotic activity in Apo-E deficient mice” J Atheroscler Thromb. 16(3):217-29.
8. Durrington P N, Mackness B, Mackness M I. Paraoxonase and atherosclerosis. Arterioscler Thromb Vasc Biol 2001; 21:473-480.
9. Frank P G, Marcel Y L. “Apolipoprotein A-I: structure-function relationships.” J Lipid Res 2000; 41:853-872.
10. Getz G S, Reardon C A. “Paraoxonase, a cardioprotective enzyme: continuing issues.” Curr Opin Lipidol 2004; 15:261-267.
11. Gillotte et al. (1996) “Apolipoprotein A-I Structural Modification and the Functionality of Reconstituted High Density Lipoprotein Particles in Cellular Cholesterol Efflux” The Journal Of Biological Chemistry 271*39):23792-23798.
12. Huuskonen J, Olkkonen V M, Jauhiainen M et al. “The impact of phospholipid transfer protein (PLTP) on HDL metabolism.” Atherosclerosis 2001; 155:269-281.
13. Jin W, Marchadier D, Rader D J. “Lipases and HDL metabolism.” Trends Endocrinol Metab 2002; 13:174-178.
14. Jonas A. “Lecithin cholesterol acyltransferase.” Biochim Biophys Acta 2000; 1529:245-256.
15. Klon A E, Segrest J P, Harvey S C. “Comparative models for human apolipoprotein A-I bound to lipid in discoidal high-density lipoprotein particles.” Biochemistry 2002; 41:10895-10905.
16. Mackness B, Durrington P N, Mackness M I. “The paraoxonase gene family and coronary heart disease.” Curr Opin Lipidol 2002; 13:357-362.
17. Mackness M I, Mackness B, Durrington P N. “Paraoxonase and coronary heart disease.” Atheroscler Suppl 2002; 3:49-55.
18. Matsunaga et al. (1999) “A novel apolipoproteins E mutation, E2 (arg25Cys), in lipoprotein glomerulopathy” Kidney International 56:421-427.
19. Maytin M, Leopold J, Loscalzo J. “Oxidant stress in the vasculature.” Curr Atheroscler Rep 1999; 1:156-164.
20. Mertens A, Holvoet P. “Oxidized LDL and HDL: antagonists in atherothrombosis.” FASEB J 2001; 15:2073-2084.
21. Moren et al. (2008) “HDL subfraction distribution of paraoxonase-1 and its relevance to enzyme activity and resistance to oxidative stress” Journal of Lipid Research, 49:1246-1253.
22. Morton R E. “Cholesteryl ester transfer protein and its plasma regulator: lipid transfer inhibitor protein.” Curr Opin Lipidol 1999; 10:321-327.
23. Navab M, Hama S Y, Hough G P et al. “High density associated enzymes: their role in vascular biology.” Curr Opin Lipidol 1998; 9:449-456.
24. Niessen H W, Krijnen P A, Visser C A et al. “Type II secretory phospholipase A₂ in cardiovascular disease: a mediator in atherosclerosis and ischemic damage to cardiomyocytes?” Cardiovasc Res 2003; 60:68-77
25. Nissen et al. (2003) “Effect of recombinant ApoA-I Milano on coronary atherosclerosis in patients with acute coronary syndromes: a randomized controlled tiral” JAMA No 5; 290(17): 2292-300.
26. PCT International Application Publication No. WO 2006/100567, published Sep. 28, 2006.
27. PCT International Application Publication No. WO 2010/093918, published Aug. 19, 2010.
28. PCT International Application Publication No. WO 2010/141097, published Dec. 9, 2010.
29. PCT International Application Publication No. WO 2012/000048, published Jan. 5, 2012.
30. Peelman F, Vandekerckhove J, Rosseneu M. “Structure and function of lecithin cholesterol acyl transferase: new insights from structural predictions and animal models.” Curr Opin Lipidol 2000; 11:155-160.
31. Phillips et al. (1997) “Predicting the Structure of Apolipoprotein A-I in Reconstituted High-Density Lipoprotein Disks” Biophysical Journal, 73:2337-2346.
32. Ryan (2010) “Nanobiotechnology applications of reconstituted high density lipoprotein” Journal of Nanobiotechnology, 8(28).
33. Segrest J P, Li L, Anantharamaiah G M et al. “Structure and function of apolipoprotein A-I and high-density lipoprotein.” Curr Opin Lipidol 2000; 11:105-115.
34. Sirtori et al. (1999) “Recombinant apolipoproteins for the treatment of vascular diseases” Atherosclerosis 142:29-40.
35. Skinner E R. (1994) “High-density lipoprotein subclasses.” Curr Opin Lipidol 5:241-247.
36. Tall A R, Lalanne F. “Phospholipid transfer protein and atherosclerosis.” Arterioscler Thromb Vasc Biol 2003; 23:1484-1485.
37. Tall A R. “Plasma cholesteryl ester transfer protein and high-density lipoproteins: new insights from molecular genetic studies.” J Intern Med 1995; 237:5-12.
38. Tselepis A D, Chapman J M. “Inflammation, bioactive lipids and atherosclerosis: potential roles of a lipoprotein-associated phospholipase A₂, platelet activating factor-acetylhydrolase.” Atheroscler Suppl 2002; 3:57-68.
39. U.S Patent Application Publication No. US 2008/0214434 A1, published Sep. 4, 2008 (Stroes).
40. U.S. patent Application Publication No. US 2011/0190193 A1, published Aug. 4, 2011 (Stroes).
41. U.S. Pat. No. 5,652,339, issued Jul. 29, 1997 (Peter Lerch et al.).
42. U.S. Pat. No. 6,559,284, issued May 6, 2003 (Hans Ageland et al.).
43. U.S. Pat. No. 7,223,726, issued May 29, 2007 (Oda et al.).
44. U.S. Pat. No. 7,491,693, issued Feb. 17, 2009 (Alphonse Hubsch et al.).
45. U.S. Pat. No. 7,759,315, issued Jul. 20, 2010 (Salvatore Cuzzocrea et al).
46. U.S. Pat. No. 7,956,035, issued Jun. 7, 2011 (Erik S. G. Stroes).
47. van Tol A. “Phospholipid transfer protein.” Curr Opin Lipidol 2002; 13:135-139.
48. Voetsch B, Loscalzo J. “Genetic determinants of arterial thrombosis.” Arterioscler Thromb Vasc Biol 2004; 24:216-229.
49. von Eckardstein et al. (1993) “Interaction of Reconstituted High Density Lipoprotein Discs Containing Human Apolipopritein A-I (ApoA-I) Variants with Murine Adipocytes and Macrophages” The Journal Of Biological Chemistry, 268(4):2616-2622.
50. von Eckardstein et al. (1994) “Physiological and clinical relevance of high-density lipoprotein subclasses.” Curr Opin Lipidol 5:404-416.
51. Zhang et al. (2010) “Recombinant high density lipoprotein reconstituted with apolipoproteins AI cysteine mutants as delivery vehicles for 10-hydroxycamptothecin” Cancer Lett. 298(1):26-33.

1. A composition comprising pegylated high-density lipoprotein (HDL) particles which comprise apolipoprotein A-I (ApoA-I), wherein at least 50% of the ApoA-I in the composition are monopegylated ApoA-I.

2. The composition of claim 1, wherein at least 90% of the ApoA-I in the composition are monopegylated ApoA-I.

3. The composition of claim 1, wherein the composition is essentially free of multipegylated ApoA-I.

4. The composition of claim 1, wherein the composition is essentially free of bile salts.

5. The composition of claim 1, wherein at least 70% of the total monopegylated ApoA-I in the composition are N-terminally pegylated.

6. (canceled)

7. The composition of claim 1, wherein the monopegylated ApoA-I comprise a polyethylene glycol covalently bonded to the ApoA-I, which polyethylene glycol has a molecular weight of between 19,000 and 21,000 Daltons.

8. (canceled)

9. The composition of claim 1, wherein the pegylated HDL particle is pegylated recombinant HDL particle or pegylated mutant HDL particle.

10. The composition of claim 1, wherein the pegylated HDL particle is pegylated reconstituted HDL particle; or
pegylated native HDL particle; or
pegylated human HDL particle.

11-12. (canceled)

13. A composition comprising pegylated high-density lipoprotein (HDL) particles which comprise pegylated proteins, wherein the pegylated proteins include at least one of apolipoprotein A-II (apo A-II), apolipoprotein A-IV (ApoA-IV), apolipoprotein A-V (Apo V), apolipoprotein C-I (Apo C-I), apolipoprotein C-II (Apo C-II), apolipoprotein C-III (Apo C-III), apolipoprotein D (Apo D), apolipoprotein E (Apo E), apolipoprotein F (Apo F), apolipoprotein M (Apo M), lecithin cholesterol acyltransferase (LCAT), cholesteryl ester transfer protein (CETP), phospholipid transfer protein (PLTP), paraoxonase, platelet-activating factor acylhydrolase (PAF-AH), clusterin (apo J), serum amyloid A (SAA) and tissue factor pathway inhibitor (TFPI).

14-15. (canceled)

16. A process for preparing a composition of claim 1 comprising pegylated HDL particles, the process comprising:
(a) admixing HDL particles with a source of polyethylene glycol to form a mixture;
(b) incubating the mixture from step (a) under conditions allowing for formation of a covalent bond between a protein of the HDL particles and a molecule of polyethylene glycol; and
(c) separating pegylated HDL particles from the product of step (b)
so as to thereby prepare the composition comprising pegylated HDL particles.

17-44. (canceled)

45. A method of treating a subject afflicted with an inflammatory vascular disease comprising administering to the subject an amount of the composition of claim 1, effective to treat the subject.

46-47. (canceled)

48. A method of treating a subject afflicted with dyslipidemia comprising administering to the subject an amount of the composition of claim 1, effective to treat the subject.

49. A method of increasing plasma HDL levels in a subject comprising administering to the subject an amount of the composition of claim 1, effective to increase plasma HDL levels in the subject.

50. A method of promoting cholesterol efflux from macrophage foam cells in a subject comprising administering to the subject an amount of the composition of claim 1, effective to promote cholesterol efflux from macrophage foam cells in the subject.

51. A method of decreasing the amount of white blood cells in a subject comprising administering to the subject an amount of the composition of claim 1, effective to decrease the amount of white blood cells in the subject.

52-60. (canceled)

61. A method of increasing the amount LCAT in a subject comprising administering to the subject an amount of a composition comprising LCAT bound to pegylated HDL particles, effective to increase the amount of LCAT in a subject.

62-67. (canceled)
